(19) United States
(12) Patent Application Publication
(10) Pub. No.: US 2005/0119532 A1
(43) Pub. Date: Jun. 2, 2005

(54) Intelligent system and method for monitoring activity and comfort

(76) Inventor: Christian Cloutier, Quebec (CA)

Correspondence Address: FULBRIGHT & JAWORSKI L.L.P., 600 CONGRESS AVE., SUITE 2400, AUSTIN, TX, 78701, US

(21) Appl. No.: 10/500,909

(22) PCT Filed: Aug. 5, 2003
(86) PCT No.: PCT/CA03/01171

(30) Foreign Application Priority Data
Aug. 5, 2002 (CA) 2397185

(51) Int. Cl. A61B 5/00; A61B 5/103; A61B 5/117

(52) U.S. Cl. 600/300; 600/595; 128/920; 128/923; 600/549; 600/534

(57) ABSTRACT

The system and the method of the present invention allows a person in charge (P) to monitor, and to act on, the activity level, physical or cerebral, and the comfort level of one ore more subject (S) simultaneously, from a distance, by the provision of a combination of sensors, at least one processing unit, a communication unit vice and at least one control unit, whereby depending on a specific needs of the subject, complementary sensors are selected to measure complementary parameters of the subject (S) and/or of the subject's environment (E) and to process and interpret these measured parameters so as to assess a level of global or localized comfort, in such a way that these parameters are available to the person in charge (P).

### FIELD OF THE INVENTION

The present invention relates to activity and comfort monitoring. More specifically, the present invention is concerned with an intelligent system and a method for monitoring activity and comfort of a subject.

### BACKGROUND OF THE INVENTION

Efforts have been made previously to create fabrics and garments that which includes an integrated infrastructure for monitoring vital signs of infants, adults or athletes for example.

For example, the published international PCT application No. WO0101855 discloses a fabric having an integrated information infrastructure, which can be incorporated or fashioned into a wearable garment and which includes a flexible infrastructure for collecting, processing, transmitting and receiving information concerning a wearer of the garment. The garment comprises a comfort component serving as a base, a plurality of signal transmission paths integrated within the comfort component, and at least one interface that provides a transmission path between the information infrastructure component that is part of the garment and the external device.

In the U.S. published patent application No. U.S. 2002/0013538A1, a method for health signs monitoring are described, including the acts of detecting at least one health sign characteristic of a person with a sensor unit that is located proximate to the individual; producing a health signal from the sensor unit that indicates at least one health sign of the individual; communicating the health signal from the individual to a receiving unit over a wireless connection; processing the health signal to determine if an emergency condition exists; and providing an indication of emergency conditions to a destination node of a network, wherein operating electrical power is applied to the receiving unit in an initialization mode, the receiving unit determining if the receiving unit has received an identification signal from the sensor unit, and receiving a health signal only from a sensor unit having the received identification signal.

However, there is still a need in the art for an intelligent system and a method for monitoring activity and comfort in a way that allows adjustment to characteristic of a subject under monitoring, and which allows an action in response to data collection and interpretation.

### OBJECTS OF THE INVENTION

An object of the present invention is therefore to provide an improved intelligent system and method for monitoring activity and comfort.

### SUMMARY OF THE INVENTION

More specifically, in accordance with the present invention, there is provided a system for monitoring activity and comfort of at least one subject, comprising at least one data acquisition unit; and at least one control unit connected to the at least one data acquisition unit, wherein the at least one data acquisition unit comprises a modular and variable set of sensors comprising a number of sensors, which nature and connection are combined according to needs of the at least one subject under monitoring.

Furthermore, the present invention provides a method for monitoring activity and comfort of at least one subject comprising the acts of collecting data, processing the collected data and transmitting the processed data to a person in charge of the at least one subject, whereby the act of collecting data uses a modular and variable set of sensors comprising a number of sensors, which nature and connection are combined according to needs of the at least one subject under monitoring, providing a continued up dating and adjustment, in an intelligent way, to the at least one subject in relation to an environment thereof.

Other objects, advantages and features of the present invention will become more apparent upon reading of the following non-restrictive description of embodiments thereof, given by way of example only with reference to the accompanying drawings.

### BRIEF DESCRIPTION OF THE DRAWINGS

In the appended drawings:

FIG. 1 is a simplified diagram of a system according to an embodiment of a first aspect of the present invention;

FIG. 2 is a flowchart of a transmission power control between the control unit and a data collecting unit of the system of FIG. 1;

FIG. 3 is a flowchart of a method according to an embodiment of a second aspect of the present invention;

FIG. 4 is a flowchart of the data collecting act of the method of FIG. 3;

FIG. 5 is a flowchart of the data processing act of the method of FIG. 3;

FIG. 6 is a flowchart of the data transmission act of the method of FIG. 3;

FIG. 7 is an isomeric simplified view of a system according to an embodiment of the present invention;

FIG. 8 is a schematic diagram of the sensing unit of the system of FIG. 7, when worn by a baby under monitoring;

FIG. 9 is an exploded view of the movement sensing unit of the system of FIG. 7; and

FIG. 10 is a schematic view of optional devices that may be added to the system of FIG. 7.

### DESCRIPTION OF EMBODIMENTS OF THE INVENTION

Generally stated, the present invention provides an intelligent system and a method allowing detection, interpretation and monitoring of levels of activity and/or comfort, of a subject, such as a person or an animal, from a distance.

Activity encompasses herein physical activity including an absence of a movement, an absence of respiration, a sleeping state, an awaken state, an active state, an intense active state or any other intermediate state, as well as a cerebral activity corresponding to each one of these states for example.

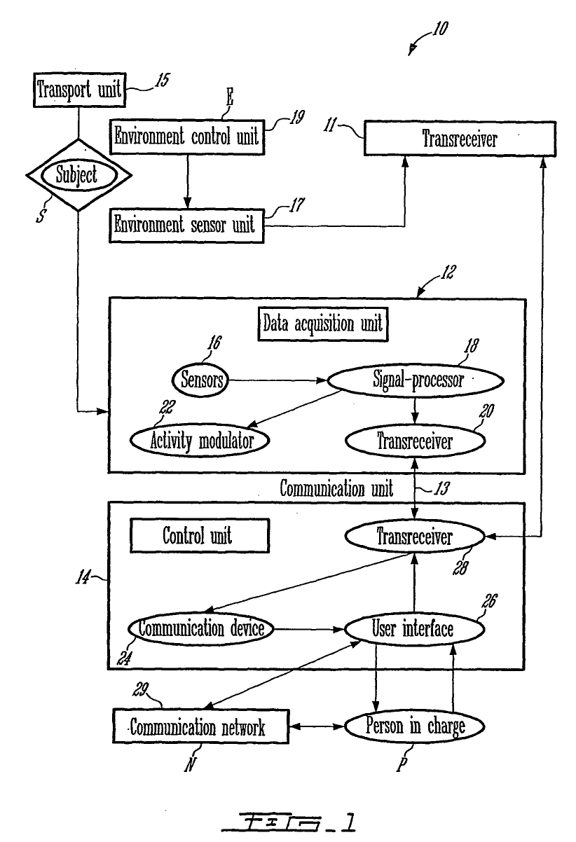

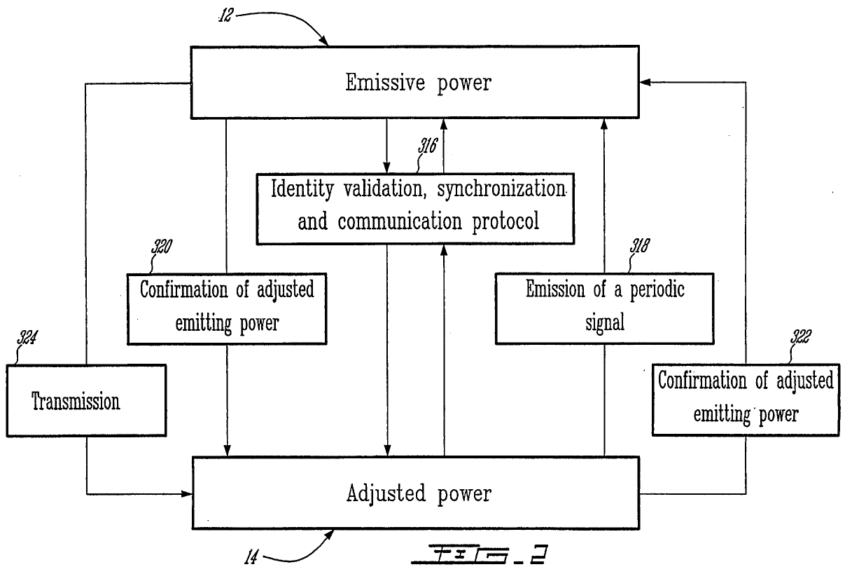

A system according to an embodiment of a first aspect of the present invention will now be described in relation to FIGS. 1-2 of the appended drawings.

As illustrated in FIG. 1, the system 10 generally comprises a data acquisition unit 12 and a control unit 14 connected by a communication unit 13.

The system 10 further comprises a transport unit 15, which allows the data acquisition unit 12 to keep track of the activity of the subject (S) under monitoring, as well as an environment unit 17, which allows keeping track of parameters of the environment (E), as will be further described hereinbelow, and a environment control unit 19, connected to the control unit 14 via a transreceiver 11 to modify measured environment parameters.

It is to be noted that the system 10 may comprise several data acquisition units 12 and several control units 14, thereby allowing, for example, a thorough context evaluation.

The data acquisition unit 12 comprises a set of sensors 16; a signal-processor 18, a transreceiver 20; and an activity modulator 22.

The set of sensors 16 is basically a modular and variable set comprising a number of sensors, wherein the nature and connection of the sensors may be varied according to a specific application, as will be exemplified further hereinbelow.

The set of sensors 16 comprises a motion sensing device, such as piezoelectric film, and/or a cerebral activity sensor.

The set of sensors 16 may further comprise a G sensor, GPS, tilt sensor, Infrared sensor, echo sensors, magnetic sensoelectrodes for example, temperature probe, moisture meter, including a sound environment or a body imaging device.

The set of sensors 16 collects parameters and sends them to the signal-processor 18, where they are interpreted to be available for display to the person in charge (P), for generating an alarm, or for activating the activity modulator 22. The transreceiver 20 allows exchange of with remote units, via the transreceiver 11 for example. The data acquisition unit 12 thus allows in time detection of characteristics of the subject (S), while the Environment sensor unit 17 collects parameters of the environment (E).

The control unit 14 comprises a communication device 24 and a user interface 26. The communication device 24 may be connected to the transreceiver 20 of the data acquisition unit 12 through a transreceiver 28. It may receive parameters of the environment (E) through a transreceiver 11 connected to the environment sensor unit 17. The communication device 24 may be an electronic circuit, connected to the user interface 26 or a wireless (RF) communication device for example. It allows a visual, sound, or tactile online consultation by the person in charge (P) or to transfer the data to a communication network (see FIG. 1). The control unit 14 may be a remote control unit.

The control unit 14 further comprises a signal processor including a memory and processing means, to store and process data received from the acquisition unit 12. As already mentioned hereinabove, it may be connected via a port 29 to the network (N) (see FIG. 1). The communication device 24 allows a person in charge (P) of the subject (S) to be informed of a status thereof, in terms of levels of activity, comfort, and of complementary parameters. The person in charge (P), in turn, may order from a distance a variation of the levels of activity or comfort, or a request for complementary data including a sound level of the environment (E), video images of the subject for instance, via the interface 26.

From the foregoing, it should now be apparent that the control unit 14 may be used by the person in charge (P) to follow in real time activity of the subject (S), to order a transmission of data and/or an activation of sensors or a retroaction.

Interestingly, the data acquisition unit 12 manages a communication setting in relation to data acquisition, while the control unit 14 is in charge of the management of the integrity and security of the system in relation to external interference for example, as well as of energy savings and lowering electromagnetic radiation by an adjustment of a power level of transmission, as will now be described.

FIG. 2 illustrates how a transmission power between the acquisition unit 12 and control unit 14 is monitored. After a step of identity validation, synchronization and communication protocol recognition (316), the control unit emits a periodic signal (act 318), so that the acquisition unit may adjust its emissive power accordingly (act 320). The control unit then confirms that the adjusted emitting power of the acquisition unit is adequate (act 322), so that transmission from the acquisition unit and the control unit may adjust to a distance and to a desired quality of transmission (act 324).

Therefore, the system of the present invention the person in charge (P) to monitor, and to act on, the activity level, physical or cerebral, and the comfort level of one ore more subject (S) simultaneously, from a distance, by the provision of a combination of sensors, at least one processing unit, a communication unit vice and at least one control unit.

The combination of sensors comprises movement sensors such as multi-wing piezofilm for example (described hereinbelow) and/or electrodes sensitive to cerebral waves, with a number of complementary sensors if required, such as a temperature sensing unit to assess the level of comfort, thereby allowing a contextual assessment of the activity and comfort.

Depending on a specific application, the complementary sensors are selected to measure complementary parameters of the subject (S) and/or of the subject's environment (E) and to process and interpret these measured parameters so as to assess a level of global or localized comfort and to be available to the remote person in charge (P).

It may be further contemplated to provide a main data acquisition unit comprising movement sensors such as a wing-shaped piezofilm and/or electrodes sensitive to cerebral waves, and a complementary data acquisition unit to collect additional complementary data.

Such a system may find application in a number of fields, such as children care, senior citizens care, either at home or in a health institution for example, security of workers etc.

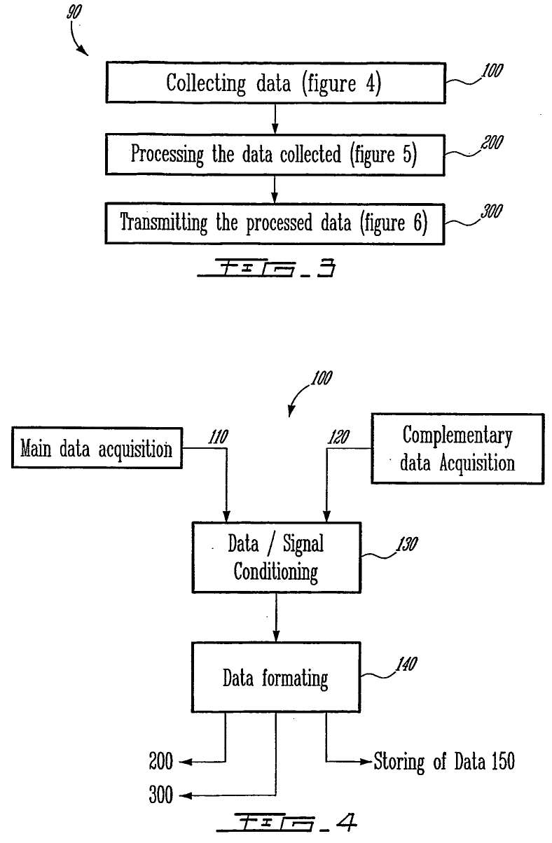

In a second aspect of the present invention, a method for monitoring activity and comfort levels is provided, as illustrated in FIGS. 3-6.

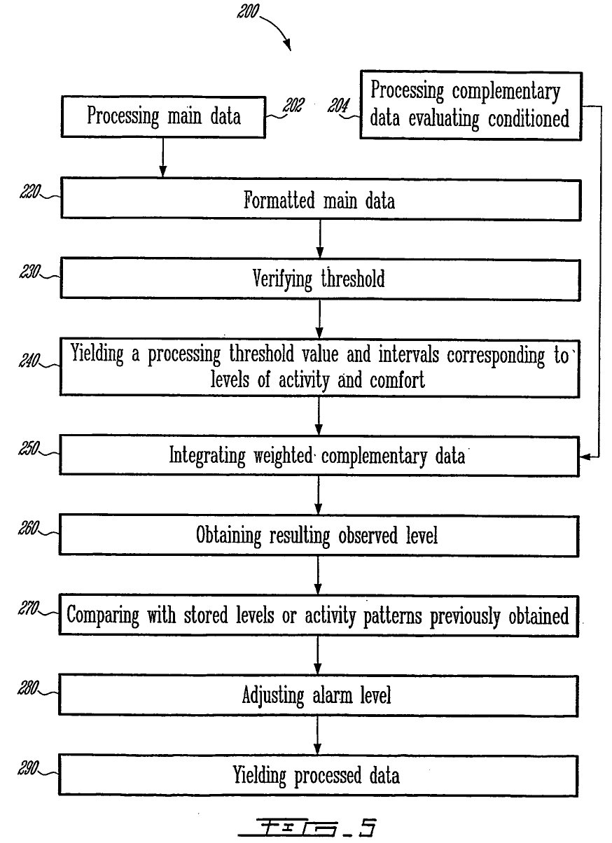

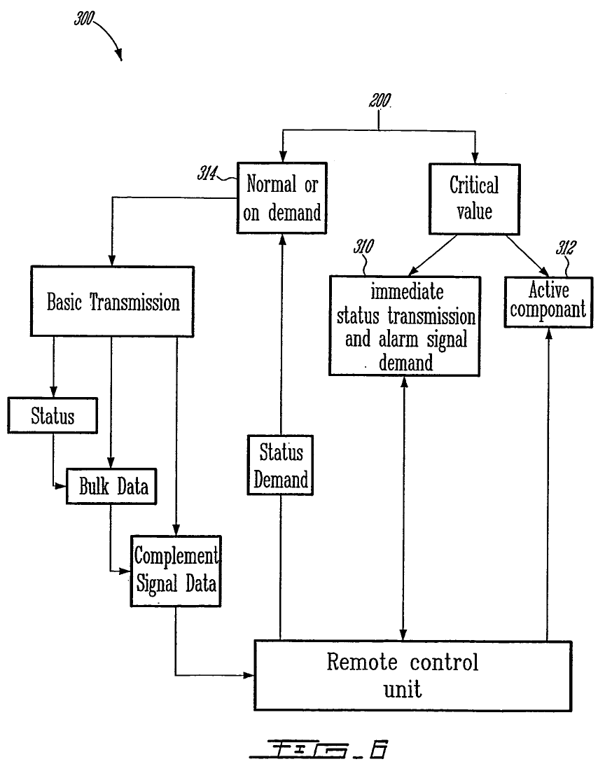

The method 90 (see FIG. 3) generally comprises collecting data (act 100, see FIG. 4), processing the data collected (act 200, see FIG. 5) and transmitting the processed data (act 300, see FIG. 6).

The collecting data step 100 is illustrated in FIG. 4. A number of data are collected by an acquisition unit, described hereinabove in relation to the first aspect of the present invention described hereinabove. Main data may be collected by a main data acquisition unit (act 110) and complementary data may be collected by a complementary data acquisition unit (act 120). The collected data are conditioned (act 130) and formatted (act 140) according to a sensibility and performance of sensors used in the collecting data steps 110, 120. The data are then stored (act 150), or directed to the processing step (act 200) or the transmitting step (act 300). As described hereinabove in relation to the first aspect of the present invention, the main data acquisition unit comprises a movement sensor and/or a cerebral activity sensor, and the complementary data acquisition unit comprises complementary sensors, which may be selected according to specific needs.

The processing step (act 200) illustrated in FIG. 5, comprises processing the main data (202), and processing the complementary data (204), in the example given hereinabove.

The conditioned and formatted main data are evaluated in relation to preset threshold values (act 220) and to criteria for threshold variation (act 230), to yield a processing threshold value and intervals corresponding to levels of activity and comfort (act 240).

The conditioned and formatted complementary data may be integrated in the evaluation, provided with a weight depending on the specific application (act 250). A resulting observed level is then obtained (act 260), which is compared with stored levels or activity patterns previously obtained (270), in order to adjust scaling of activity levels and alarm levels (act 280) and to yield processed data (act 290).

Thus, the processing step (act 200) involves an analysis of the data collected in act 100 in such a way as to allow a on-going adjustment to the activity characteristics and history of the subject (S), for example in the case of a baby, to adjust to the baby's growth and a corresponding increase in movements intensity and frequency. Therefore, the method of the present invention allows a continued up dating and adjustment, in an intelligent way, to the subject (S) and the environment (E) under monitoring.

As illustrated in FIG. 6, in the case when the data processing act 200 indicates that a threshold is reached, a transmission to the control unit (act 310) is immediately initiated), and a retroaction unit 312 such as a physical activity stimulator, connected to the processing unit, may be immediately activated (act 320). The data processing step therefore allows an adjustment in real time to specific characteristics of activity and comfort of a given subject.

When the analysis of the data (act 200) indicates that the parameters monitored are in an acceptable range, in relation to threshold values for example, a basic transmission of the data to the control unit is performed (act 314).

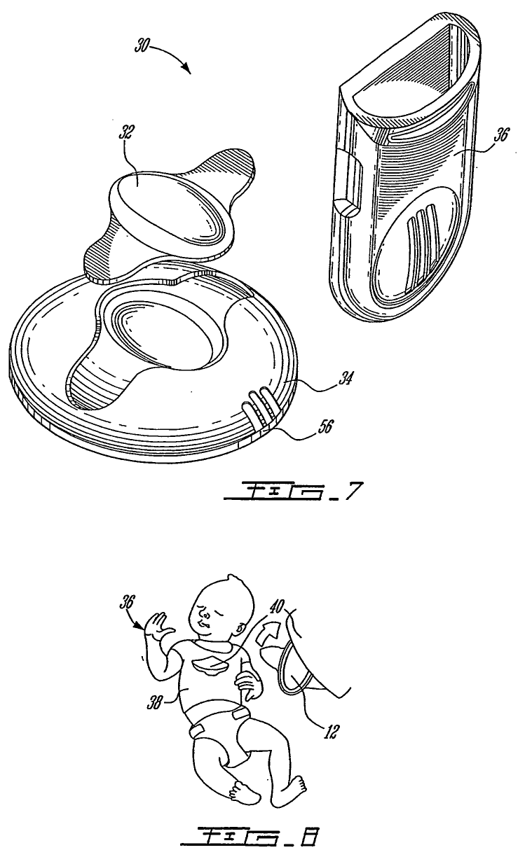

As a way of example, a specific application of the present invention will now be described, in relation to FIGS. 7-10 of the appended drawings. This example relates to an activity/comfort intelligent monitoring system and method for a baby.

The system 30 comprises a sensing unit 32 provided with a base 34, and a control unit 36.

The sensing unit 32 is typically of a very small size, and portable, and includes a number of sensors and a transmitter means; it is designed to be worn by the baby under monitoring. The base 34 is used for transportation of the sensing unit 32, and may also provide for a recharge thereof.

The control unit 36 may be of a pager type and is typically provided with a screen. It is designed to be held by a person in charge of the baby.

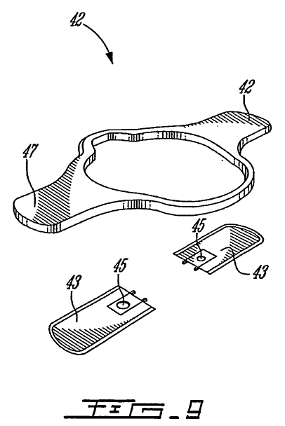

As illustrated in FIG. 8, the sensing unit 32 may be maintained is a close relationship with the body of the baby 36 under monitoring, for example by means of a tight piece of clothing 38, which may be provided with a pocket 40 to receive the sensing unit 32. Alternatively, a strip of cloth or strap may be used instead of the piece of clothing 38 to maintain the sensing unit 32 is a closed relationship with the body of the baby 36 under monitoring.

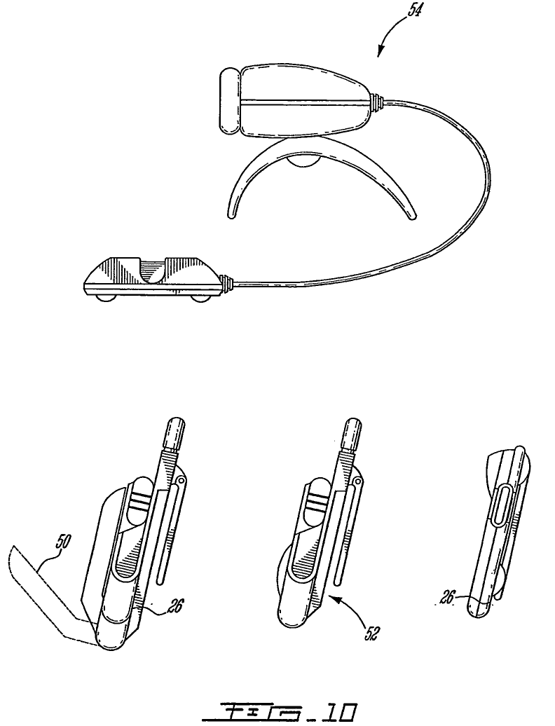

The sensing unit 32 may comprise a wing-shaped piezoelectric device 42, best seen in FIG. 9, allowing sensing movements of the body of the baby 38 by a bending and distortion of wings 43 that generate an electrical current. The resulting electrical current is amplified and transmitted to a micro controller for analysis and comparison with activity thresholds corresponding to a sleeping state of the baby for instance. Thus, the level of movements of the baby 38 is interpreted and transmitted, or first transmitted and then interpreted, to the control unit 36, for example by microwave transmission. It is to be noted that activity thresholds are updated to take into account changes in the baby's condition, such as its age and size for example.

More precisely, the wing-shaped piezoelectric device 42 illustrated in FIG. 9 comprises at least one piezo film coated with a flexible, non-allergenic and isolating material, and is provided for example with two wings 43. The wings 43 are provided with perforations 45 allowing calibrated position thereof. The flexible, non-allergenic and isolating material is molded so as to embed the piezo films with a wing shape, thereby protecting the piezo films and providing a seal ring between sides of the base 34 (see FIG. 7).

When located close to the sternum of the baby, such a device 42 allows detecting movements of its rib cage and of diaphragm, and determining its respiratory movements. By processing differential signals from the two wings 43 and analysis of the frequency and intensity of these electrical signals, its heartbeat may be obtained. Therefore, the device 42 allows assessing an activity level of the baby.

Interestingly, such a device 42 may be integrated in a portable assembly 47 and does not require any fixation means, since it may be secured into place in a pocket of a cloth worn by the subject or held by a strap around its body. Its allows detecting movements on a surface it is in contact with in a plurality of sites.

The control unit 36 receives a signal of the state of the baby at intervals, so that the person (P) in charge of the baby 38 may be alerted by a sound alarm or a visual alarm appearing on the screen of the control unit 36.

A number of alarms may be determined, including for example an alarm corresponding to awaking of the baby, an alarm corresponding to an absence of movement after a predetermined delay, etc.

The control unit 36 may be programmed to allow the person in charge of the baby 38 to check the baby's state at any time, and therefore to take adequate actions using an activity modulator as described hereinabove.

Adequate actions may comprise for example an action performed by the person in charge (P) or programmed by the person in charge (P) following the reception of the interpreted data. They may include activating a transmission, activating one of the sensors of the set of sensors of the data acquisition unit, a request for a audio or a video signal in the environment or towards remote receivers, modifications of the activity and/or comfort parameters detected or request for modifications thereof sent to separate systems via a user interface (including stimulation, nature modification and decrease of an activity parameter for example, and heating, ventilating, light modification, positioning modifications in the comfort parameters for example, etc.).

Indeed, the control unit 36 may be provided with a screen for display of icons giving information related to the activity of the baby, such as its resting state, its awaken state, its active awaken state and an absence of movement thereof, including an absence of respiration thereof, or its being in a prone position for example.

In order to detect that the baby's movements have resulted in a prone position, the sensing unit 32 may be provided with a tilt sensor, in such the prone position is detected by a microcontroller and triggers the sending of a corresponding signal to the control unit 36. An associated alarm signal, either sound or visual, may also be triggered.

A possible functionality of the system 30 is to monitor the temperature of the baby 38. Therefore, the sensing unit 32 allows measuring a cutaneous temperature of the baby 38 and comparing it to a reference temperature set as a comfort temperature zone so as to determine whether the baby 38 is comfortable. The comfort temperature zone is predetermined according to the baby's specific characteristics, including for example its age, its size, and a proper sensitivity thereof, its state of health etc., and may be updated accordingly when required.

The control unit 36 allows the person in charge of the baby 38 to check icons related to the baby's temperature.

Alternatively, a thermistor device may be positioned in the base 34 of the sensing unit 32, to measure the cutaneous temperature via an opening in the pocket 40 illustrated in FIG. 8. This temperature is then compared to a predetermined comfort temperature zone, which is stored in a memory of the system. An indicator may indicate whether the measured temperature lies in the predefined comfort zone, allowing the person in charge to take adequate actions.

Obviously, the reference comfort temperature is a predetermined temperature value that is programmed in the system 20 that may be updated according to individual variations of the baby 38. A predetermined range considered as a physiologic range may be preset to limit the amount of allowed updating for example.

In the case when no movement has been detected for a while, for example, 20 seconds or any other predetermined lapse of time, a stimulating device connected to the baby 38, such as a vibrating motorized device that may be directly contacting the baby's body, may be triggered with selectionable level of stimulation by the person in charge through the control unit 36, to cause the baby 38's reaction.

Interestingly, the use of wing-shaped piezofilm sensors 42 illustrated in FIG. 9 also allows monitoring heart beating by a differential sensing of the wings. Depending on a detected frequency, the heart beating may be interpreted as characteristic of a rest state or of an active state and yields a signal delivered to the control unit 36. The control unit 36 may be provided with an indicator lamp for example, which flickering frequency allows visualizing variation of the heart beating detected.

A failure of the transmission between the sensing unit 32 and the control unit 36 may be immediately reported by the same heart beating indicator lamp or another indicator lamp remaining on and/or a sound alarm, for example. A same mechanism may be used to indicate battery status for example.

The control unit 36 may further comprise sound module or audio/video module 50 in such a way as to be used as a bi-directional intercommunicating unit or to be able to receive image, of the type “clip on” 52 or “slot” (see FIG. 10). Correspondingly, the base 34 is then provided with sound and image devices.

The base 34 may be provided with a microphone, a loudspeaker 56 (see FIG. 7), a radio receiver and a radio transmitter, or with a camera 54 and a video transmitter for example, the control unit 36 being then equipped with complementary devices, such as a sound transmitter to allow the person in charge (P) to speak to the baby 38 via the loudspeaker 56 of the base 34 for example, in an upgradable fashion, as illustrated in FIG. 10. Any sound or video signal may then be exchanged between the base 34 and the control unit 36 upon request.

The system 30 allows an identification link between the sensing unit 32 and the control unit 36, in such a way that the sensing unit 32 is uniquely connected to the control unit 36. The control unit 36 may however be used in connection to a number of different sensing units 32, to allow a same person to supervise a number of babies, for example.

The base 34 may be used for recharging the sensing unit 32, and also the control unit 36 by allowing a contact to a modular loader and a rechargeable battery provided as an option, wherein the rechargeable battery may be plugged to the electrical network by means of an external current transformer adapted to the network and working at a low voltage. Alternatively, the control unit 36 may be powered by batteries. In any case, warning indictors are provided to notify a user of failing batteries.

From the foregoing, it should now be apparent that the system and method of the present invention allows for a supervision and modification, even from a distance, of a level of physical and/or cerebral activity and of comfort, by using and combining sensors and communication devices providing a detection, characterization, follow-up and modification of the level of physical activity and of comfort of a living person or of an animal in particular. Physical activities include a state of immobility (i.e. absence of a movement), a sleeping state, a more or less active awaken state, a more or less intense active state and any variant in between these extremes, as well as a level and nature of a cerebral activity. Comfort is assessed as a subjective perception and/or as a parameterized perception of specific parameters including temperature, humidity, stress points of the body, as well as global comfort direct or indirect measurements including stress indications and brain waves.

In a contemplated application related to senior citizen, G sensor and tilt sensor may be used to detect a fall of the subject; the system and method of the present invention allow monitoring activity levels before and after a falling event, thereby establishing an historical record of data and allowing a post event analysis of movement, and enable a remote monitoring wherein a non-response situation triggers an alarm.

Although specific applications of the system and method of the present invention have been presented, people in the art will appreciate that they may find application in a number of fields, including for example monitoring of a level of activity and work intensity of a labor force or of people whose activity places them at risk and requiring a possibility of a remote intervention, such as a firefighter, a security guard, or a mine worker for instance. Moreover, the system and method of the present invention may also be contemplated for supervision of animals, for detection of animal kindling and for animal supervision purposes for example.

Although the present invention has been described hereinabove by way of embodiments thereof, it can be modified, without departing from the nature and teachings of the subject invention as defined in the appended claims.

1. A system for monitoring activity and comfort of at least one subject, comprising:
at least one data acquisition unit comprising a modular and variable set of sensors comprising a number of sensors, which nature and connection are combined according to needs of the at least one subject under monitoring; and
at least one control unit connected to said at least one data acquisition unit;
wherein said at least one data acquisition unit further comprises devices selected in the group consisting of a microphone, a loudspeaker, a radio receiver, a radio transmitter, a camera and a video transmitter; and
wherein said at least one control unit comprises modules selected in the group consisting of sound modules and audio/video modules.

2. The system according to claim 1, wherein said set of sensors comprises main sensors selected in the group consisting of movement sensors and cerebral activity sensors, and complementary sensors selected in the group consisting of G sensor, GPS, tilt sensor, infrared sensor, echo sensors, magnetic sensoelectrodes, temperature probe, moisture meter, sound environment sensor and body imaging device.

3. The system according to claim 1, wherein said at least one data acquisition unit further comprises a signal-processor receiving data from said set of sensors, and an activity modulator connected to said signal-processor.

4. The system according to claim 1, wherein said at least one control unit comprises a communication device and a user interface.

5. The system according to claim 4, wherein said communication device comprises a signal processor including a memory and processing means.

6. The system according to claim 4, wherein said communication device is connected to a network.

7. The system according to claim 4, wherein said communication device allows adjusting a transmission power between the data acquisition unit and the control unit.

8. The system according to claim 1, further comprising a transport unit, which allows said at least one data acquisition unit to keep track of the activity of the at least one subject, and an environment unit which allows keeping track of environmental parameters.

9. A system for monitoring activity and comfort of at least one subject, comprising:
at least one data acquisition unit comprising a modular and variable set of sensors comprising a number of sensors, which nature and connection are combined according to needs of the at least one subject under monitoring; and at least one control unit connected to said at least one data acquisition unit;
wherein said set of sensors comprises a wing-shaped piezoelectric sensor and sensors selected in the group consisting of G sensor, GPS, tilt sensor, infrared sensor, echo sensors, magnetic sensoelectrodes, temperature probe, moisture meter, sound environment sensor and body imaging device.

10. The system according to claim 9, wherein said wing-shaped piezoelectric sensor comprises at least one piezo film coated with a flexible, non-allergenic and isolating material.

11. The system according to claim 9, wherein said wing-shaped piezoelectric sensor detects movements selected in the group consisting of the at least one subject's rib cage movements, the at least one subject's diaphragm movements, the at least one subject's respiratory movements and the at least one subject's heartbeat.

12. The system according to claim 9, wherein said wing-shaped piezoelectric sensor comprises wings which position is able to be calibrated.

13. The system according to claim 4, wherein said user interface allows a person in charge of the at least one subject to be informed of a state thereof, to order a retroaction according to the state of the at least one subject and to request complementary data selected in the group consisting of sound levels and video images.

14. The system according to claims 2 and 9, wherein the temperature probe allows measuring a cutaneous temperature of the subject and comparing it to a reference temperature set as a comfort temperature zone.

15. The system according to claim 14, wherein the comfort temperature zone is updated in relation to characteristics of the at least one subject selected in the group consisting of its age, its size, a proper sensitivity thereof, its state of health.

16. The system according to anyone of claims 2 and 9, wherein the set of sensors is maintained in a close relationship with the at least one subject's body.

17. The system according to anyone of claims 1 and 9, wherein said at least one control unit receives signals of a state of the at least one subject at intervals and provides alerts.

18. The system according to claim 17, wherein the alarms are triggered by states selected in the group consisting of awaking of the at least one subject, absence of movement after a predetermined delay and position variations.

19. The system according to anyone of claims 1 and 9, wherein activity comprises activities selected in the group consisting of rest, physical activity, absence of a movement, absence of respiration, a sleeping state, an awaken state, an active state, an intense active state, an intermediate active state and cerebral activity, and monitoring comprises an assessment of an intensity and of a nature of the activity from a distance.

20. The system according to any one of claims 1 and 9, wherein said data acquisition unit is integrated in a portable assembly.

21. A system for monitoring activity and comfort, comprising:
at least one data acquisition unit comprising a modular and variable set of sensors; and
at least one control unit connected to said at least one data acquisition unit;
wherein said set of sensors comprises at least one sensor selected in the group consisting of a G sensor and a tilt sensor to detect a fall of the subject.

22. The system according to claim 21, said system being used for monitoring activity levels before and after a falling event, thereby establishing an historical record of data and allowing a post event analysis of movement, said system allowing a remote monitoring wherein a non-response situation triggers an alarm.

23. A method for monitoring activity and comfort using the system according to anyone of claims 1, 9 and 21.
